(19) United States
(12) Patent Application Publication
(10) Pub. No.: US 2026/0249261 A1
(43) Pub. Date: Aug. 27, 2026

(54) PORTABLE CONTAINER HOLDER FOR USE IN A BIOPROCESS

(71) Applicant: Sartorius Stedim Biotech GmbH, Goettingen (DE)

(72) Inventor: Marc Hogreve, Adelebsen (DE)

(21) Appl. No.: 19/160,350

(22) PCT Filed: Feb. 14, 2024
(86) PCT No.: PCT/EP2024/053741

§ 371 Date: Aug. 27, 2025

(30) Foreign Application Priority Data
Feb. 28, 2023 (DE) 10 2023 104 889.9

(51) Int. Cl. B01F 33/502; B01F 33/453; B01F 35/513; B01F 101/44; C12M 1/00; C12M 1/06; C12M 3/00

(52) U.S. Cl. CPC B01F 33/5024; B01F 33/4533; B01F 35/513; C12M 23/26; C12M 23/28; C12M 23/46; C12M 23/52; C12M 27/02; B01F 2101/44

(57) ABSTRACT

Portable container mounting for use in a bioprocess for holding and transporting a single-use container which is filled with a fluid and has received a magnetically driven mixing element for mixing the fluid, wherein the portable container mounting has a container receptacle for receiving the single-use container having a drive location on which the mixing element is able to be disposed in the single-use container received in the container receptacle in such a manner that the mixing element is able to be driven by a drive unit. The portable container mounting has a transport safeguard for securing and releasing the mixing element, which is disposed on the drive location, in its position and that the transport safeguard has a magnet which is movable between a defined secure position, in which the mixing element is positionally secured, and a defined release position, in which the mixing element is positionally released.

### CROSS-REFERENCE TO RELATED APPLICATIONS

This application is a national stage application under 35 U.S.C. 371 of International Patent Application Serial No. PCT/EP2024/053741, entitled “Portable Container Holder for Use in a Bioprocess,” filed Feb. 14, 2024, which claims priority from German Patent Application No. DE 10 2023 104 889.9, filed Feb. 28, 2023, the disclosure of which is incorporated herein by reference.

### FIELD OF THE TECHNOLOGY

Embodiments relate to a portable container mounting for use in a bioprocess for holding and transporting a single-use container filled with a fluid, to a system for use in a bioprocess, and to a method for holding and transporting a single-use container.

### SUMMARY

Portable container mountings for holding and transporting single-use containers are used in various embodiments in different fields of biotechnology. For instance, in order to be able to store, transport or process a specific fluid, such as, for example, a fluid containing a purified pharmaceutical substance, or a cultivation medium, the fluid is first filled into an, in particular flexible, single-use container. While the single-use container per se would be able to be transported only with complexity and able to be held with difficulty, in particular due to the extremely high weight of the received fluid, the single-use container is disposed in a portable container mounting, typically prior to filling with the fluid. Holding and transporting the single-use container can be enabled by the portable container mounting in particular for the frequent case when the single-use containers accommodate large quantities of fluid. The portable container mounting can be lifted for transport, for example, for instance by means of a lifting truck, and be moved or directly displaced, for instance by way of transport rollers or rails.

A container mounting for holding and transporting a single-use container filled with fluid first has a container receptacle. A single-use container, which can be configured so as to correspond to the container receptacle, is able to be received in the container receptacle.

Because it is often provided while the single-use container filled with fluid is held that the fluid is also mixed within the single-use container, the single-use container receives a mixing element, such as, for instance, an agitator, in particular an impeller. The mixing element is magnetically driven in such a way that the mixing element can be driven in a contactless manner and the fluid can be mixed within the single-use container when required.

The container receptacle has a drive location by way of which the mixing element is able to be magnetically driven by a drive unit. The drive unit can be, for instance, an external drive unit which is likewise able to be disposed on or in the proximity of the drive location. The mixing element in the single-use container is disposed on the drive location when the single-use container is received in the container receptacle. Driving the mixing element by way of the drive location by the drive unit then takes place, for example, in a contactless manner by way of a magnetic field which is generated by the drive unit and sets the mixing element within the single-use container in rotation.

WO 2017/129203A1 . from which some embodiments proceed, relates to a portable container mounting which enables holding and transporting a single-use container filled with fluid, wherein the fluid here is able to be mixed by way of a mixing element received by the single-use container during holding. The container mounting has a container receptacle in which the single-use container is able to be received. The container receptacle has a drive location at which the mixing element is able to be disposed in such a way that the mixing element can be driven at the drive location by an external drive unit.

While portable container mountings of this type from the prior art for holding and transporting a single-use container filled with fluid have indeed proven successful and the fluid can also be reliably mixed therewith during holding, this nevertheless results in the disadvantage that the magnetically driven mixing element can slide out of position, in particular during transport. A type of bearing mandrel on which the mixing element is initially rotatably disposed is indeed typically provided, but the mixing element can slide down from this bearing mandrel and thereupon move freely within the single-use container. For this reason, it can easily occur, for example in the event of shocks or movements of the fluid, that the mixing element which has initially been disposed on the drive location, in particular on the bearing mandrel, slides out of position within the single-use container and thereupon is no longer disposed on the drive location. Consequently, the mixing element which has slid out of position is also no longer able to be driven, and the fluid is thus no longer able to be mixed.

In principle, some solutions are indeed known from the prior art, such as a magnetic cap from US 2005/0002274A1 , which are intended to avoid the above-mentioned problem, but these solutions are presently not fully developed, in particular in terms of their handling. Thus, it is typically the case for instance with a screw-on or attachable cap that the latter is lost, is no longer able to be found when required, or is able to be assembled and disassembled only with difficulty.

Various embodiments are based on the problem of optimizing the known portable container mounting in terms of holding and transporting a single-use container which has received a mixing element, in particular to optimize same to such an extent that the mixing element is able to be reliably held in position, in particular also during transport.

The above problem is addressed by various features described herein.

A consideration that the portable container mounting has a transport safeguard is essential. The mixing element can be reliably held in position during holding the single-use container as well as during transport of the single-use container by way of the transport safeguard. In the process, the magnetically driven, and thus also magnetic, mixing element is positionally secured by a magnet of the transport safeguard when the magnet is moved to a defined secure position. In the secure position, the magnetic field of the magnet acts on the mixing element and correspondingly holds the latter in position within the single-use container. As soon as the mixing element is to be released from its position, the magnet is able to be moved to a defined release position in which the magnetic field of the magnet no longer positionally secures the mixing element. This may be the case, for instance, when the mixing element is to be driven by way of an external drive unit and the fluid is to be mixed. The positionally released mixing element is in particular able to be driven by the drive unit in such a way that the mixing element rotates. As a result, the fluid can correspondingly be mixed within the single-use container.

In detail, it is proposed that the portable container mounting has a transport safeguard for securing and releasing the mixing element, which is disposed on the drive location, in its position, and that the transport safeguard has a magnet which is able to be moved between a defined secure position, in which the mixing element is positionally secured, and a defined release position, in which the mixing element is positionally released.

According to some embodiments, the transport safeguard has a magnet mounting. As a result of the magnet mounting, the magnet can be held and moved in a structurally simple manner. The magnet mounting can have a magnet guide which enables a guided, thus defined, movement of the magnet. A guide fastening of the magnet mounting represents a structural possibility for rigidly fastening the magnet guide to the container receptacle.

An operator assembly of the transport safeguard is advantageously specified in some embodiments. The transport safeguard is able to be operated by way of the operating assembly in such a way that the magnet can be moved to the release position or secure position when required. An operating element of the operating assembly can enable a comfortable manual activation of the operating assembly, in particular by hand. The magnet can be reliably locked in the secure position or the release position by way of a locking assembly, such that said magnet is not able to be moved unintentionally. A preload assembly can preload the magnet so as to move the latter automatically to the secure position or release position by way of the preload. As a result, the operating comfort can be increased, on the one hand, and user errors can be avoided, on the other hand, by ensuring that the magnet is moved to the corresponding position.

Various embodiments to the magnet of the transport safeguard. The design embodiment of the magnet as a permanent magnet represents a favorable design embodiment of the magnet with comparatively little complexity. In comparison to a design embodiment of the magnet as a solenoid, for instance, this moreover results in the advantage that the magnet configured as a permanent magnet is independent of any potential current sources, and the operational reliability of the transport safeguard can thus be increased, in particular during transport. A magnet clearance of the magnet can enable the magnet to be disposed about the mixing element, as a result of which reliable position securing is achieved. Moreover, the magnet clearance can increase the operating comfort of the transport safeguard, in that the latter is configured as a positioning aid.

According to various embodiments, the container receptacle has one or a plurality of container walls and one container base on which the drive location is disposed. As a result, a structurally simple container receptacle can be achieved. The drive location can have a drive clearance, as a result of which reliable driving of the mixing element by the drive unit is ensured.

Advantageous assemblies of the magnet, which enable reliable position securing of the mixing element, are proposed in some embodiments.

Various embodiments provide aspects of the structural design embodiment of the profitable container mounting. A drive receptacle offers a structurally favorable manner of receiving an external drive unit so as to be operationally ready. Transport rollers ensure the portability of the container mounting, wherein said transport rollers enable in particular that the portable container mounting is able to be moved by a user without excessive input of force without further aids, such as a lifting truck or the like.

According to various embodiments, a system for use in a bioprocess is provided. The system has a portable container mounting having a container receptacle, and a single-use container which has received a magnetically driven mixing element for mixing a fluid, wherein the single-use container is received in the container receptacle, the container receptacle has a drive location, and the mixing element in the single-use container received in the container receptacle is disposed on the drive location of the container receptacle in such a manner that the mixing element is able to be driven, in particular magnetically, by a drive unit.

It is essential in the system according to the proposal that the portable container mounting has a transport safeguard for securing and releasing the mixing element, which is disposed on the drive location, in its position, wherein the transport safeguard has a magnet which is movable between a defined secure position, in which the mixing element is positionally secured, and a defined release position, in which the mixing element is positionally released. Reference may be made to all explanations pertaining to the portable container mounting according to the proposal.

Advantageous design embodiments of the single-use container and of the mixing element received by the latter. A flexible single-use container can offer advantages, in particular in the non-filled state, during transport and storage and production. An agitator, in particular an impeller, can ensure uniform mixing of the fluid within the single-use container and at the same time be able to be driven in a contactless manner.

According various embodiments, the system has a drive unit. The drive unit enables the magnetically driven mixing element, in particular the agitator, such as the impeller, to be driven in a contactless manner. In particular the design embodiment of the drive unit separate from the portable container mounting enables the flexible use of the drive unit in a multiplicity of portable container mountings.

According to various embodiments, a method for holding and transporting a single-use container, while using a portable container mounting, is provided. In the method, the single-use container is received in a container receptacle of the portable container mounting, and a magnetically driven mixing element for mixing a fluid, which mixing element is received in the single-use container, is disposed on a drive location of the container receptacle in such a manner that the mixing element is able to be driven by a drive unit.

It is essential here that the mixing element disposed on the drive location is secured in its position by a transport safeguard of the portable container mounting, which transport safeguard has a magnet, in that the magnet is moved, in particular from a defined release position, in which the mixing element is positionally released, to a defined secure position, in which the mixing element is positionally secured.

Reference may be made to all explanations pertaining to the portable container mounting according to the proposal and the system according to the proposal.

According to various embodiments, the mixing element can be positionally released, for instance in order to be able to drive the mixing element by way of a drive unit.

Various embodiments relate to advantageous possibilities for moving the magnet between the different positions.

Advantageous design embodiments which improve the operating capability of the transport safeguard, in that the magnet is automatically moved from the secure position to the release position or from the release position to the secure position are proposed in some embodiments.

Various embodiments provide a portable container mounting for use in a bioprocess for holding and transporting a single-use container which is filled with a fluid and has received a magnetically driven mixing element for mixing the fluid, wherein the portable container mounting has a container receptacle for receiving the single-use container having a drive location on which the mixing element is able to be disposed in the single-use container received in the container receptacle in such a manner that the mixing element is able to be driven by a drive unit on the drive location, wherein the portable container mounting has a transport safeguard for securing and releasing the mixing element, which is disposed on the drive location, in its position, and in that the transport safeguard has a magnet which is movable between a defined secure position, in which the mixing element is positionally secured, and a defined release position, in which the mixing element is positionally released.

In various embodiments, the transport safeguard has a magnet mounting for holding the magnet in the secure position and the release position, and in that the magnet is movable rotationally and/or translationally, in particular linearly, by way of the magnet mounting from the secure position to the release position and/or from the release position to the secure position. In some embodiments, the magnet mounting has a magnet guide for the guided movement of the magnet between the secure position and the release position, and in that the magnet guide has a guide bearing and a motion element which is mounted so as to be movable in the guide bearing. In some embodiments, the magnet mounting has a guide fastening for rigidly fastening the magnet guide to the container receptacle.

In various embodiments, the transport safeguard has an operating assembly for operating the transport safeguard, and in that the magnet is movable manually and/or automatically by way of the operating assembly from the secure position to the release position and/or from the release position to the secure position. In some embodiments, the operating assembly has an operating element, in particular a handle, and/or in that the operating assembly has a locking assembly, in particular having a locking element, for locking the magnet in the secure position and/or the release position, and/or in that the operating assembly has a preloading assembly, in particular having a spring element, for preloading the magnet to the secure position or the release position.

In various embodiments, the magnet is configured as a permanent magnet, and/or in that the magnet has a magnet clearance which, in the secure position of the magnet, at least partially surrounds the mixing element in the single-use container received in the container receptacle, in some embodiments in that the magnet clearance is configured as a positioning aid for the easier positioning of the magnet on the drive location and/or about the mixing element, in some embodiments the magnet clearance is configured in such a manner that the magnet has a cross section which is horizontal in particular in the secure position and represents substantially a circle, a segment of a circle, in particular a semi-circle, a horseshoe, an ellipse or a partial ellipse.

In various embodiments, the container receptacle is formed from one or a plurality of container walls and one container base, and in that the drive location is disposed on the container base, in some embodiments the drive location has an, in particular substantially circular or elliptic, drive clearance which extends over the entire thickness of the container base and in which the mixing element is able to be disposed at least partially or the drive unit is able to be disposed at least partially.

In various embodiments, the magnet is disposed in the secure position on the drive location, in particular so as to be coaxial with the drive clearance. In some embodiments, the magnet is disposed in the secure position on one side on the drive location disposed on the container base.

In various embodiments, the portable container mounting has a drive receptacle for receiving the drive unit, which is disposed below the container receptacle, which drive receptacle is in particular delimited upwardly by the container base of the container receptacle. In some embodiments, the drive receptacle is formed at least from a plurality of supports and/or plates, which drive receptacle is delimited at least on two sides, in particular on three sides, by the plurality of supports and/or plates, and/or in that the portable container mounting has a transport assembly having a plurality of transport rollers for displacing the portable container mounting. In some embodiments, the transport rollers are disposed on the drive receptacle, in particular on the supports and/or plates.

Various embodiments provide a system for use in a bioprocess, wherein the system has a portable container mounting, in particular a portable container mounting as provided herein, having a container receptacle and a single-use container which has received a magnetically driven mixing element for mixing a fluid, wherein the single-use container is received in the container receptacle, the container receptacle has a drive location, and the mixing element is disposed in the single-use container received in the container receptacle on the drive location of the container receptacle in such a manner that the mixing element is able to be driven by a drive unit, wherein the portable container mounting has a transport safeguard for securing and releasing the mixing element, which is disposed on the drive location, in its position, and in that the transport safeguard has a magnet which is movable between a defined secure position, in which the mixing element is positionally secured, and a defined release position, in which the mixing element is positionally released.

In various embodiments, the single-use container is configured as a flexible single-use container, in particular in the manner of a flexible pouch, and/or to be sterile, and/or in that the mixing element has an, in particular rotationally symmetrical, agitator, such as an impeller.

In various embodiments, the system has a drive unit. In some embodiments, the drive unit is configured separately from the portable container mounting and is able to be disposed on the drive location when required. In some embodiments, the drive unit is able to be received by a drive receptacle of the portable container mounting, and in that the drive unit is able to be disposed in the drive receptacle or able to be removed from the drive receptacle in that the drive unit and/or the portable container mounting is movable in a sliding and/or pulling manner.

Various embodiments provide a method for holding and transporting a single-use container while using a portable container mounting, in particular a portable container mounting as provided herein, wherein the single-use container is received in a container receptacle of the portable container mounting, and a magnetically driven mixing element for mixing a fluid, which is received in the single-use container, is disposed on a drive location of the portable container mounting in such a manner that the mixing element is able to be driven by a drive unit, wherein the mixing element disposed on the drive location is secured in its position by a transport safeguard of the portable container mounting, which transport safeguard has a magnet, in that the magnet is moved, in particular from a defined release position, in which the mixing element is positionally released, to a defined secure position, in which the mixing element is positionally secured.

In various embodiments, the mixing element disposed on the drive location is positionally released by the transport safeguard of the portable container mounting in that the magnet is moved, in particular from the defined secure position, to a defined release position.

In various embodiments, the magnet is moved in a pivoting manner and/or translationally, in particular linearly, between the secure position and the release position. In some embodiments, the magnet is moved manually and/or automatically between the secure position and/or the release position.

In various embodiments, the magnet is moved from the secure position to the release position when a drive unit is received by a drive receptacle of the portable container mounting, in particular is pushed or retracted into the drive receptacle, and/or in that the magnet is moved from the release position to the secure position when the drive unit is removed, in particular is extracted or pushed out, from the drive receptacle, and/or in that the magnet is moved from the secure position to the release position when a drive unit is received by the drive receptacle, in particular is pushed or retracted into the drive receptacle, and/or in that the magnet is moved from the release position to the secure position before a drive unit is removed in particular is extracted or pushed out, from the drive receptacle.

### BRIEF DESCRIPTION OF THE DRAWINGS

Various aspects will be explained in more detail hereunder by means of a drawing which only shows exemplary embodiments. In the drawing

FIG. 1 shows a portable container mounting having a transport safeguard in a) a perspective illustration, b) a fragment, which shows the transport safeguard in a secure position, and c) a fragment, which shows the transport safeguard in a release position,

FIG. 2 shows the transport safeguard according to FIG. 1 in a) the secure position, wherein the position of a drive location of the container mounting is indicated with dashed lines, and b) the release position in a perspective view,

FIG. 3 shows a second exemplary embodiment of a transport safeguard in a) the secure position, wherein the position of a drive location is indicated with dashed lines, and b) the release position in a perspective view,

FIG. 4 shows a further exemplary embodiment of a transport safeguard in a) the secure position, wherein the position of a drive location is indicated with dashed lines, and b) the release position in a perspective view, and

FIG. 5 shows the portable container mounting according to FIG. 1 having a received single-use container and in a) without a received drive unit and the transport safeguard in the secure position and b) having a received drive unit and the transport safeguard in the release position in a lateral sectional view.

### DETAILED DESCRIPTION

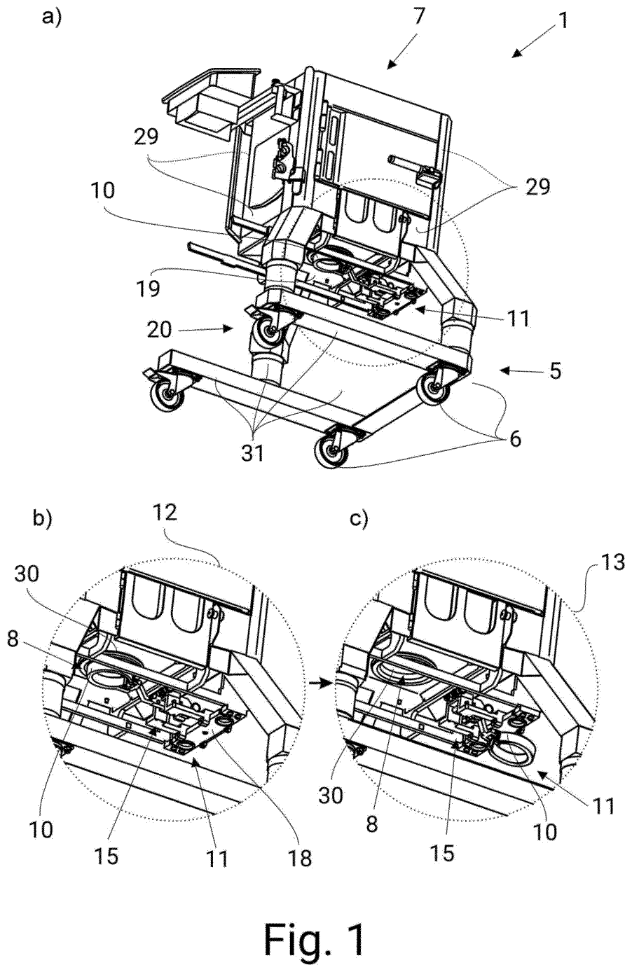

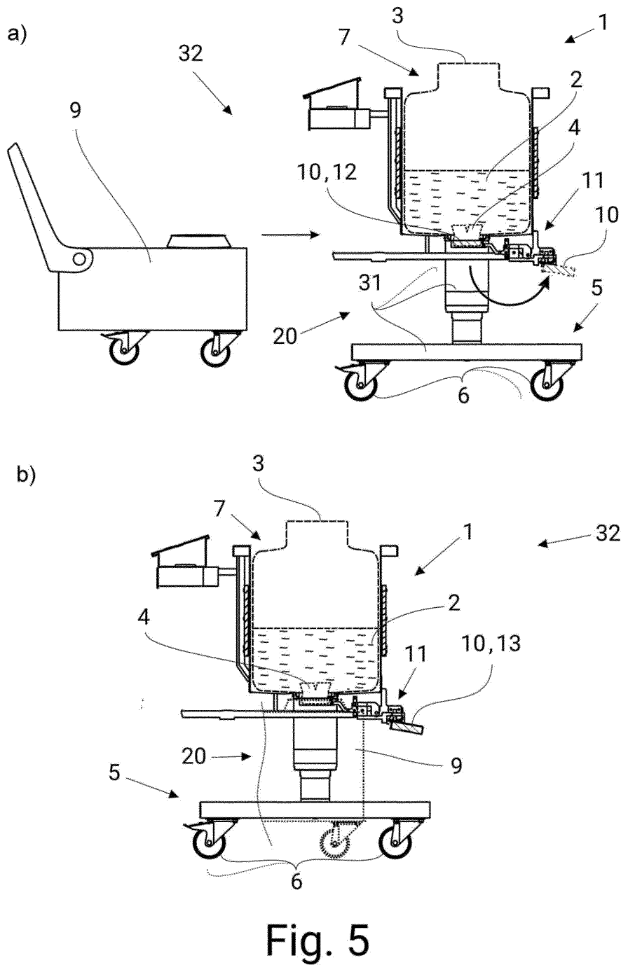

The exemplary embodiment, which is illustrated in FIGS. 1a), 1b), 1c) and 5a), 5b), relates to a portable container mounting 1 for holding and transporting a single-use container 3 which is filled with fluid 2, in particular a liquid. The single-use container 3 can in particular be a single-use container 3 which is able to be used in bioprocesses. The single-use container 3 can be configured to be flexible, in particular in the manner of a pouch, so that the external volume thereof increases when filled with fluid 2. The fluid 2 can in particular be a fluid 2 used in bioprocesses, for instance a fluid 2 containing a purified pharmaceutical substance, or a cultivation medium.

The single-use container 3 has received a magnetically driven mixing element 4. The mixing element 4 can be received so as to be freely rotatable in the single-use container 3. The fluid 2 within the single-use container 3 can be mixed by the mixing element 4, for instance when a substance is added to the fluid 2 or in order to homogenize the fluid 2. It is possible that the fluid 2 is mixed by way of the mixing element 4 while the single-use container 3 is held by means of the portable container mounting 1.

In the context of this application, “portable” means that the container mounting per se is able to be transported. Transporting here can take place with a received single-use container 3 as well as without a received single-use container 3. In order to transport the container mounting, the latter can be lifted, for example by means of a lifting truck, and displaced. It is also possible that the container mounting has a transport assembly 5 having transport rollers 6, as can be derived from FIG. 1a), for instance, by way of which the container mounting is displaceable.

The portable container mounting 1 according to the proposal has a container receptacle 7 for receiving the single-use container 3. The single-use container 3 can in particular be disposed empty in the container receptacle 7 and subsequently be filled with fluid 2. The portable container mounting 1 can be configured to be substantially cuboid or cube-shaped, as is depicted by way of example in FIG. 1a). The single-use container 3 which is filled with fluid 2, is received in the container receptacle 7 and takes up the space in the latter, is depicted by way of example in FIGS. 5a), 5b). It is possible in particular that the single-use container 3 is not completely filled with fluid 2, but has air in a head region, for instance.

The container receptacle 7 has a drive location 8 at which the mixing element 4 in the single-use container 3 received in the container receptacle 7 is able to be disposed in such a manner that the mixing element 4 is able to be driven, in particular magnetically, by a drive unit 9 at the drive location 8. The mixing element 4 which is received by the single-use container 3, thus disposed in the interior of the single-use container 3, can be driven in a contactless manner by way of the drive location 8. This can take place in that an, in particular external, drive unit 9 sets the mixing element 4 in rotation by way of a magnetic field, so that the fluid 2 is correspondingly mixed. While mixing takes place, the mixing element 4 within the single-use container 3 remains in contact with the fluid 2. However, the mixing element 4 can be driven in a contactless manner by way of the drive unit 9, thus without contact between the mixing element 4 and the drive unit 9.

During holding, in particular mixing, and transporting by the portable container mounting 1, the mixing element 4 remains in the single-use container 3. The magnetically driven 10 mixing element 4 is in principle freely movable within the single-use container 3 and is not per se mounted so as to be in a fixed position by the single-use container 3 in the interior of the single-use container 3. In principle, it is indeed possible that the mixing element 4 is received in the single-use container 3 and is rotatably mounted at a specific position, for example in a type of depression, by a type of bearing mandrel, or similar, but the mixing element 4 is not positionally fixed at this position within the single-use container 3. In this context, even after the mixing element 4 is disposed on the drive location 8, there would fundamentally be the risk of the mixing element 4 sliding out of position within the single-use container 3, for example when said mixing element falls out of the above-mentioned depression, jumps off the bearing mandrel, or similar.

It is therefore essential in the portable container mounting 1 according to the proposal that the portable container mounting 1 has a transport safeguard 11 for securing and releasing the mixing element 4, which is disposed on the drive location 8, in particular within the single-use container 3, in its position, and that the transport safeguard 11 has a magnet 10 which is able to move between a defined secure position 12, in which the mixing element 4 is positionally secured, in particular by the magnetic field of the magnet 10, and a defined release position 13, in which the mixing element 4 is positionally released.

It is reliably ensured by the transport safeguard 11 of the portable container mounting 1 that the mixing element 4 received by the single-use container 3 is able to be held in position. As a result, the mixing element 4 can be positionally fixed, for example in the above-mentioned depression, on the bearing mandrel, or similar. It can thus be avoided that the mixing element 4 slides out of position in particular during transport in which shocks may arise, for example. In the defined secure position 12 (FIG. 1b), the magnet 10 of the transport safeguard 11 ensures by way of a magnetic field that the magnetically driven 10 mixing element 4 is held in position within the single-use container 3 and is correspondingly positionally secured. However, in order to be able to still drive the mixing element 4 by way of a drive unit 9, for instance, the magnet 10 of the transport safeguard 11 can be moved to the defined release position 13 (FIG. 1c) when required, in which the mixing element 4 is correspondingly positionally released and able to be driven as well as rotated. Positionally securing the mixing element 4 is generally not required during mixing, because the mixing element 4 can be held in position anyway by the magnetic field of the drive unit 9.

In the context of the application, “positionally secured” to this extent includes that the mixing element 4 received by the single-use container 3 is held in a specific position, in particular disposed on the drive location 8, by the magnet 10 of the transport safeguard 11. In the defined secure position 12 of the magnet 10, the magnetic field of the latter acts on the mixing element 4.

In the context of the application, “positionally released” by contrast includes that the mixing element 4 is correspondingly not held in position by the magnet 10 of the transport safeguard 11. In the defined release position 13 of the magnet 10, the magnetic field of the latter, in some embodiments, does not act on the mixing element 4 or only weakly on the mixing element 4 in such a manner that the latter is not held in position by the magnet 10.

In the context of this application and the positions, “defined” includes that the secure position 12 and the release position 13 are established positions to which the magnet 10 is able to be repeatedly moved. The “defined secure position” 12 can thus be in particular a specific position in which the magnetic field of the magnet 10 acts on the mixing element 4, or else also be an angular range in which the magnetic field of the magnet 10 acts on the mixing element 4. The “defined release position” 13 can in particular be a specific position in which the magnetic field of the magnet 10 does not act, or only weakly acts, on the mixing element 4 in such a manner that the mixing element 4 is not held in position, or else also be an angular range in which the magnetic field of the magnet 10 does not act, or only weakly acts, on the mixing element 4 in such a manner that the mixing element 4 is not held in position.

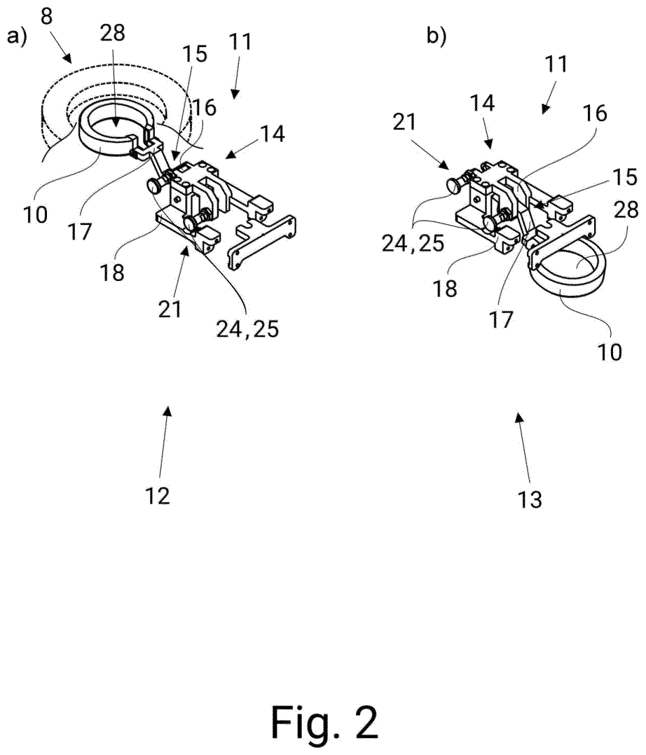

An exemplary embodiment of a transport safeguard 11 of the portable container receptacle 7 is in each case depicted in FIGS. 2a), 2b), 3a), 3b) and 4a), 4b). In FIGS. 2a), 3a) and 4a), the respective transport safeguard 11 is located in the secure position 12, and in FIGS. 2b), 3b) and 4b) in the release position 13. The transport safeguards 11 are correspondingly illustrated to be disassembled in the figures, but can be assembled when required, as is derived from FIGS. 1 and 5a), 5b), for instance. The position of a potential drive location 8 of a container mounting 1 is illustrated with dashed lines in FIGS. 2a), 3a) and 4a).

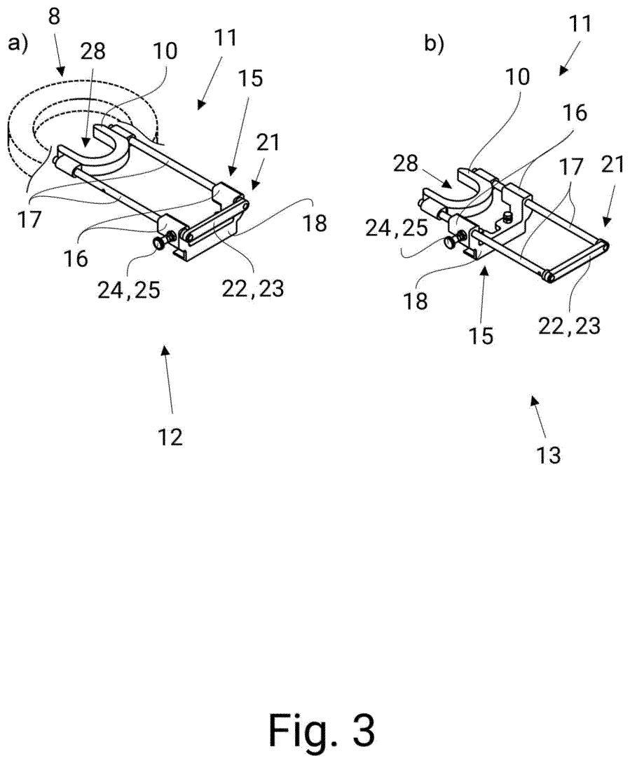

It can be provided that the transport safeguard 11 has a magnetic mounting 14 for holding the magnet 10 in the secure position 12 and the release position 13. The magnet 10 is able to be moved rotationally and/or translationally, in particular linearly, by way of the magnet mounting 14 from the secure position 12, depicted by way of example in FIGS. 2a), 3a) and 4a), to the release position 13, depicted by way of example in FIGS. 2b), 3b) and 4b), and/or from the release position 13 to the secure position 12. The magnet 10 can be moved rotationally by way of the magnet mounting 14 by a pivoting movement, as implemented in the exemplary embodiments of FIG. 2a), 2b) or 4a), 4b), or is moved translationally, in particular linearly, by a sliding movement, as implemented in the exemplary embodiment of FIGS. 3a), 3b). It is also possible that the magnet 10 is able to be moved rotationally as well as translationally, in particular linearly, in that said magnet is pivoted and displaced, for example, wherein these movements can take place in particular simultaneously, at least partially simultaneously or successively. The magnet 10 is disposed on the magnet mounting 14.

In the exemplary embodiments depicted, the magnet mounting 14 has a magnet guide 15 for the guided movement of the magnet 10 between the secure position 12 and the release position 13, wherein the magnet guide 15 has a guide bearing 16 and a motion element 17 which is movably mounted in the guide bearing 16. The motion element 17 can be mounted rotationally, as depicted by way of example in FIG. 2a), 2b) or 4a), 4b), or linearly movably, as depicted by way of example in FIGS. 3a), 3b), in the guide bearing 16. It is possible that the magnet mounting 14 has two movably mounted motion elements 17, as is illustrated by way of example in FIGS. 3a), 3b). In various embodiments, the guide bearing 16 has two guide clearances in which the, in particular cylindrical, motion elements 17 are movably mounted in the direction of the main axis and/or in which the, in particular cylindrical, motion elements 17 are mounted parallel to one another. In various embodiments, the magnet 10 can be disposed, in particular rigidly, on one side on the motion element 17 or the motion elements 17. The magnet 10 can in particular be disposed on the motion element 17 or the motion elements 17 by way of one or a plurality of clamping elements, such as clamps, for instance, or threaded elements.

For rigidly fastening the magnet guide 15 to the container receptacle 7, the magnet mounting 14 can have a guide fastening 18 for rigidly fastening the magnet guide 15 to the container receptacle 7, as can be seen from FIGS. 1b) and 2a), 2b), for instance. It is possible that the guide fastening 18 is able to be screwed and/or clamped to the container receptacle 7, for example by way of threaded bolts or clamping means. In various embodiments, the magnet mounting 14 is disposed by way of the guide fastening 18 on a container base 19 of the container receptacle 7, in particular so as to face the ground. The guide fastening 18 can be disposed rigidly relative to the container receptacle 7 or a drive receptacle 20, the latter being discussed in more detail hereunder. The magnet 10 can be able to be moved relative to the container receptacle 7 and/or the drive receptacle 20 by way of the magnet mounting 14.

The transport safeguard 11 particularly can have an operating assembly 21 for operating the transport safeguard 11. The magnet 10 is able to be moved, in particular manually and/or automatically, by way of the operating assembly 21 from the secure position 12 to the release position 13 and/or from the release position 13 to the secure position 12. The manual and/or automatic activation will be discussed in yet more detail hereunder in the context of the method according to the proposal.

For manually activating the operating assembly 21, it is conceivable that the operating assembly 21 has an operating element 22. In the exemplary embodiment of FIGS. 3a), 3b), the operating element 22 is configured as a handle 23 which is able to be activated by a user, in particular using one hand. In various embodiments, the operating element 22 can be disposed on the end on one or a plurality of motion elements 17 and/or, in various embodiments, connect two motion elements 17 to one another.

In the context of the operating assembly 21, it has proven to be advantageous in particular that the operating assembly 21 has a locking assembly 24 for locking the magnet 10 in the secure position 12 and/or the release position 13. The locking assembly 24 can in particular have one locking element 25 or a plurality of locking elements 25. It is possible that the locking assembly 24 has two locking elements 25, wherein the magnet 10 is able to be locked by way of one of the locking elements 25 in the secure position 12 and the magnet 10 is able to be locked by way of the other one of the locking elements 25 in the release position 13. The locking element 25, or the locking elements 25, can in each case be configured, in some embodiments, as latching bolts which are able to be activated manually, in particular using one hand, as is the case in the exemplary embodiments of FIGS. 2a), 2b) and 3a), 3b), for instance. The locking element 25, or the locking elements 25, can in each case be preloaded to a locking position in such a way that the magnet 10, when reaching the secure position 12 and/or the release position 13, is automatically locked by the locking element 25 or by the locking elements 25. The locking assembly 24, in particular the locking element 25 or the locking elements 25, can in particular be able to be unlocked manually, such as by hand by a user.

Alternatively or additionally, the operating assembly 21 can have a preloading assembly 26, in particular having a spring element 27, for preloading the magnet 10 to the secure position 12 or the release position 13. It is conceivable that the spring element 27 is configured as a torsion spring or as a tension spring or as a compression spring. By way of the preloading assembly 26, the magnet 10 can be moved automatically from the secure position 12 to the release position 13 or from the release position 13 to the secure position 12, for example when the locking assembly 24, in particular the locking element 25 or the locking elements 25, are unlocked by a user. It is conceivable in particular that the magnet 10 is moved manually from the secure position 12 to the release position 13 and automatically from the release position 13 to the secure position 12 by the preloading assembly 26. Alternatively, it is possible that the magnet 10 is moved automatically from the release position 13 to the secure position 12 by the preloading assembly 26 and manually from the secure position 12 to the release position 13.

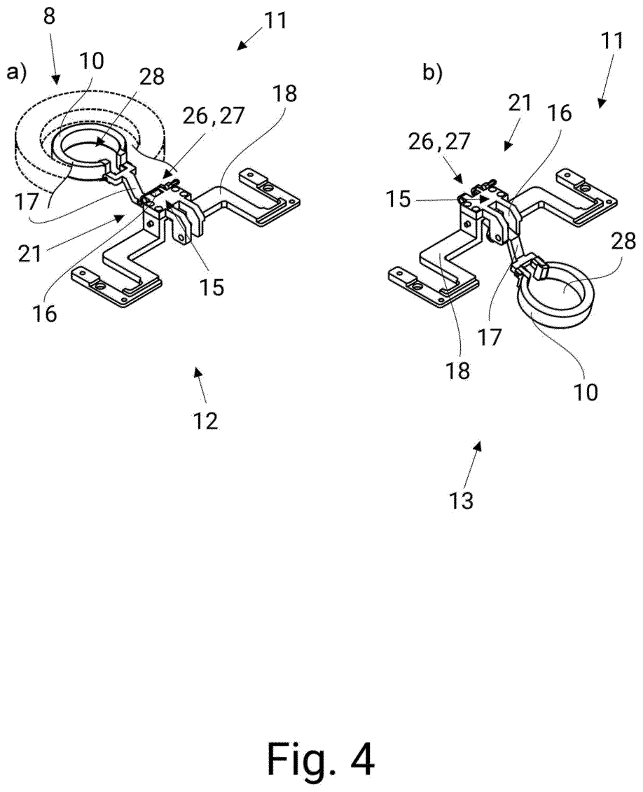

In various embodiments, it is provided that the magnet 10 is configured as a permanent magnet. The magnet 10 can comprise iron, cobalt and/or nickel as material. Alternatively or additionally, the magnet 10 has a magnet clearance 28. In the secure position 12 of the magnet 10, the magnet clearance 28 can at least partially surround the mixing element 4 in the single-use container 3 received in the container receptacle 7, as is derived from FIG. 5a), for example. In the context of the design embodiment of the magnet 10, it is conceivable that the magnet clearance 28 is configured in such a manner that the magnet 10 has a cross section which is horizontal in particular in the secure position 12 and substantially represents a circle, a segment, as illustrated by way of example in FIGS. 2a), 2b) and 4a), 4b), a horseshoe, as illustrated by way of example in FIGS. 3a), 3b), an ellipse or a partial ellipse. The magnet 10 can in particular be configured to be open on one side, in particular along its circumference. It can be conceivable that the magnet 10 in the secure position 12 at least partially surrounds the mixing element 4, in particular the agitator, such as an impeller, for instance.

The magnet clearance 28 can be configured to be circular, as illustrated by way of example in FIGS. 2a), 2b) and 4a), 4b). The magnet clearance 28 can be configured as a positioning aid for the simplified positioning of the magnet 10 on the drive location 8 and/or about the mixing element 4. In various embodiments, the magnet clearance 28 is configured so as to correspond to the drive location 8 and/or the mixing element 4.

In terms of the container receptacle 7, it can be provided that the container receptacle 7 has one or a plurality of, in some embodiments at least four, container walls 29 and one container base 19. The drive location 8 is disposed on the container base 19. In the exemplary embodiment of FIG. 1a), two of the four container walls 29 are in each case disposed so as to be substantially perpendicular to one another. The container base 19 can delimit the container receptacle 7 on one side. The container receptacle 7, in some embodiments, can be configured to be open on one side, so that a single-use container 3 is able to be received in the container receptacle 7. It is conceivable in particular that the single-use container 3 is able to be covered by a cover and the container receptacle 7 is closed by the cover on the open side. In various embodiments, at least one of the container walls 29 has a closable side door or is formed by such a side door. The container receptacle 7 can be configured so as to be substantially cuboid, in particular cube, as is depicted by way of example in FIG. 1a).

Alternatively or additionally, it is conceivable that the drive location 8 has an, in particular substantially circular or elliptic, drive clearance 30 which extends across the entire thickness of the container base 19 and in which the mixing element 4 is able to be at least partially disposed or the drive unit 9 or the magnet 10 is able to be at least partially disposed. By way of example, the circular drive clearance 30 can be seen in FIG. 1c).

It can be provided that the magnet 10 is disposed in the secure position 12 on the drive location 8, in particular so as to be coaxial with the drive clearance 30. In FIG. 5a), the magnet 10 is located in the secure position 12. The mixing element 4, in particular the agitator, such as the impeller, of the single-use container 3 is disposed on the drive location 8. In this way, the magnet 10 which is likewise disposed in the secure position 12 on the drive location 8 secures the mixing element 4, in particular the agitator, such as the impeller, and prevents the latter from sliding out of position within the single-use container 3, for instance during transport. In this way, the mixing element 4, in particular the agitator, such as the impeller, is positionally secured. As depicted in FIG. 5a), the magnet 10 is disposed, in particular so as to face the ground, in the secure position 12 on one side on the drive location 8 disposed on the container base 19. It is possible that the magnet 10 bears on the container base 19 in the secure position 12. In various embodiments, the magnet 10 is aligned horizontally in the secure position 12.

In order to be able to drive the mixing element 4 by an external drive unit 9, it can be provided and for instance in the exemplary embodiment according to FIGS. 1a) and 5a), 5b) that the portable container mounting 1 has a drive receptacle 20 for receiving the drive unit 9, which is disposed below the container receptacle 7. The drive receptacle 20 is configured in such a manner that the drive unit 9 is able to be disposed in the latter in such a way that the mixing element 4 is able to be driven correspondingly. In structural terms, it is possible that the drive receptacle 20 is delimited upwardly by the container base 19 of the container receptacle 7, as can be seen from FIG. 1a), for example.

In terms of the portability of the container mounting, it has been found to be extremely advantageous that the portable container mounting 1 has a transport assembly 5 having a plurality of transport rollers 6 for displacing the portable container mounting 1. As a result of the transport rollers 6, the container mounting can be moved in a rolling manner, for instance to transport the single-use container 3 received in the container receptacle 7. In the exemplary embodiment of FIG. 1a), the transport assembly 5 has four transport rollers 6. At least some, in particular two, of the transport rollers 6 can be able to be locked. Owing to this fact, the portable container mounting 1 can be prevented from being unintentionally moved. In various embodiments, it is provided that the drive receptacle 20 is formed at least from a plurality of supports 31 and/or plates, wherein the drive receptacle 20 is delimited at least on two sides, in particular on three sides, by the plurality of supports 31 and/or plates. The supports 31 can enable the load bearing capability. In various embodiments, the supports 31 comprise a plurality of vertical supports, which are disposed vertically, and a plurality of horizontal supports. In the exemplary embodiment according to FIG. 1a), the drive receptacle 20 is formed from three vertical supports and three horizontal supports. It is conceivable that one plate or a plurality of plates are disposed on the supports 31, for instance to protect the drive unit 9, which is able to be received in the drive receptacle 20, against external influences. The transport rollers 6 can be disposed on the drive receptacle 20, in particular on the supports 31 and/or plates. As can be seen from FIG. 1a), the drive receptacle 20 is configured so as to be free of the base. As a result, a drive unit 9 can be moved, for instance in a sliding manner, into the drive receptacle 20. In various embodiments, the drive receptacle 20 is open at least on one side.

Besides the portable container mounting 1 according to the proposal, a system 32 for use in a bioprocess is moreover proposed, wherein the system 32 has a portable container mounting 1, in particular a portable container mounting according to the proposal, having a container receptacle 7 and a single-use container 3 which has received a magnetically driven mixing element 4 for mixing a fluid 2, wherein the single-use container 3 is received in the container receptacle 7, the container receptacle 7 has a drive location 8, and the mixing element 4 is disposed in the single-use container 3 received in the container receptacle 7 on the drive location 8 of the container receptacle 7 in such a manner that the mixing element 4 is able to be driven, in particular magnetically, by a drive unit 9.

In the system 32 according to the proposal it is essential that the portable container mounting 1 has a transport safeguard 11 for securing and releasing the mixing element 4, which is disposed on the drive location 8, in its position, wherein the transport safeguard 11 has a magnet 10 which is able to be moved between a defined secure position 12, in which the mixing element 4 is positionally secured, and a defined release position 13, in which the mixing element 4 is positionally released. A system 32 according to the proposal is illustrated by way of example in FIGS. 5a), 5b).

Reference may be made to all explanations pertaining to the portable container mounting 1 according to the proposal.

It can be provided that the single-use container 3 is configured as a flexible single-use container 3. The flexible single-use container 3 is dimensionally unstable. By receiving the single-use container 3 in the container receptacle 7, the single-use container 3 is held and is able to be filled with fluid 2. The single-use container 3 filled with fluid 2 comes to bear on the container receptacle 7 and is held in such a way as can be seen by way of example from FIGS. 5a), 5b). In various embodiments, the single-use container 3 is configured in the manner of a flexible pouch. Alternatively or additionally, the single-use container 3 is configured to be sterile. This can be significant for use in particular in the field of biotechnology in that contaminations of the fluid 2 received in the single-use container 3 are avoidable. The single-use container 3 can in particular be configured as a bioreactor and/or a cultivation container. It can be provided that the mixing element 4 is configured as an agitator, in particular as an impeller. The mixing element 4 configured as an impeller is depicted in FIG. 5b). The mixing element 4, in particular the agitator, such as the impeller, in some embodiments, is configured without a shank and/or without a shaft. The mixing element 4, in particular the agitator, such as the impeller, can in particular be configured to be rotationally symmetrical. It is conceivable that the mixing element 4, in particular the agitator, such as the impeller, has an integral housing in which in particular a permanently magnetic material and/or a magnetic material, such as iron, cobalt or nickel, is received.

In various embodiments, the system 32 has a drive unit 9. The drive unit 9 can be configured as illustrated in FIG. 5b) as an external drive unit 9 which can be configured separately from the portable container mounting 1, thus as a stand-alone unit. The drive unit 9 can be able to be disposed on the drive location 8 when required. The drive unit 9 can be able to be received by the drive receptacle 20 of the container mounting. In this context, it can be possible that the drive unit 9 is able to be disposed in the drive receptacle 20 or able to be removed from the drive receptacle 20, in that the drive unit 9 and/or the portable container mounting 1 is able to be moved in a sliding and/or pulling manner. In FIG. 5b), the drive unit 9 is able to be moved into the drive receptacle 20 or out of the drive receptacle 20 by sliding or pulling.

Furthermore proposed is a method for holding and transporting a single-use container 3 while using a portable container mounting 1, in particular a portable container mounting according to the proposal, wherein the single-use container 3 is received in a container receptacle 7 of the portable container mounting 1 and a magnetically driven mixing element 4 for mixing a fluid 2, which is received in the single-use container 3, is disposed on a drive location 8 of the portable container mounting 1 in such a manner that the mixing element 4 is able to be driven by a drive unit 9.

It is essential in the method according to the proposal that the mixing element 4 disposed on the drive location 8 is secured in its position by a transport safeguard 11 of the portable container mounting 1, which transport safeguard 11 has a magnet 10, in that the magnet 10 is moved in particular from a defined release position 13, in which the mixing element 4 is positionally released, to a defined secure position 12, in which the mixing element 4 is positionally secured.

The portable container mounting 1 used in the method can in particular be part of a system 32 according to the proposal.

Reference may be made to all explanations pertaining to the portable container mounting 1 according to the proposal and to the system 32 according to the proposal.

In the method it can be provided that the position of the mixing element 4 disposed on the drive location 8 is positionally released by the transport safeguard 11 of the portable container mounting 1, in that the magnet 10 is moved to a defined release position 13. The magnet 10 can be moved so as to reciprocate between the secure position 12 and the release position 13. In the release position 13, the mixing element 4 is able to be driven. In this way, the fluid 2 received in the single-use container 3 can be mixed.

In terms of the movement of the magnet 10, it can be provided that the magnet 10 is moved in a pivoting manner between the secure position 12 and the release position 13. This is the case, for instance, in the transport safeguard 11 depicted in FIGS. 2a), 2b) and 4a), 4b). Alternatively, it is likewise conceivable that the magnet 10 is moved translationally, in particular linearly, between the secure position 12 and the release position 13. In the embodiment of the transport safeguard 11 according to FIGS. 3a), 3b), the magnet 10 is movable linearly between the secure position 12 and the release position 13. It is likewise conceivable that the magnet 10 is moved in a pivoting manner and translationally, in particular linearly. In the process, the magnet 10 can first be displaced linearly and subsequently pivoted, for example.

It has been demonstrated to be particularly preferable in some embodiments that the magnet 10 is moved manually and/or automatically between the secure position 12 and/or the release position 13.

In the context of this application, “manually” includes movements in which the movement of the magnet 10 takes place, in particular completely, by a user. The user can move the magnet 10 directly, for instance by way of the operating assembly 21, in that said user pulls or pushes, for example, the operating element 22 or pivots the magnet 10 by way of the operating element 22, or can move the magnet 10 indirectly by way of the operating assembly 21, in that said user moves, for example, the magnet 10 by way of the operating assembly 21 when inserting or retracting the drive unit 9 into the drive receptacle 20.

In the context of this application, “automatically” includes movements which take place by a motor or a preloading assembly 26, in particular a spring element 27, without the user carrying out the movement. The movements can be initiated by the user, for instance by a control command triggered by the user or by unlocking the locking assembly 24 by a user. Thus, in the simplest case it is conceivable, for instance, that the magnet 10 is moved manually from the secure position 12 to the release position 13 and from the release position 13 to the secure position 12. The operating assembly 21, in some embodiments, can be configured without a preloading assembly 26, and the magnet 10 cannot be preloaded. In this case, the magnet 10 can be moved manually, in particular exclusively, by way of an operating assembly 21, in particular an operating element 22, between the secure position 12 and the release position 13.

The manual movement can, very generally, take place by hand by a user or take place by disposing or removing the drive unit 9. Thus, it is conceivable for example in the transport safeguard 11 according to FIGS. 3a), 3b) that the user pulls the motion element 17 configured as a handle 23, in order to move the magnet 10 from the secure position 12 to the release position 13, and pushes the motion element 17 configured as the handle 23, in order to move the magnet 10 from the release position 13 to the secure position 12.

The manual movement can, very generally, take place likewise by disposing or removing the drive unit 9, thus by inserting or retracting or sliding out or extracting the drive unit 9 into or from the drive receptacle 20. Thus, by way of example, it is possible that the user activates the operating assembly 21 by way of the drive unit 9, in that the drive unit 9 is pushed, for instance, against the operating element 22 during disposal or removal, and the operating element 22 is correspondingly operated. For this purpose, the operating element 22 can be configured, for instance, as a pressure rail in which the drive unit 9 engages or which the drive unit 9 activates by pressure.

As an alternative to the purely manual activation, it is also possible that the magnet 10 is moved manually from the secure position 12 to the release position 13 and automatically from the release position 13 to the secure position 12. The magnet 10 can be preloaded in the direction of the secure position 12 by the preloading assembly 26. It is conceivable here that the magnet 10 is moved manually from the secure position 12 to the release position 13 in that the operating assembly 21, in particular the operating element 22, is activated by hand by a user or by way of the drive unit 9 when the latter is being disposed by a user. The magnet 10 is moved automatically from the release position 13, in particular due to the preload of the preloading assembly 26, to the secure position 12, for example when the drive unit 9 is removed, in particular pushed or extracted, from the drive receptacle 20 and/or when a locking assembly 24, in particular a locking element 25, is unlocked. As a result, it can be ensured that the mixing element 4 is automatically positionally secured, for instance when the drive unit 9 is removed from the drive receptacle 20.

Alternatively, it is likewise conceivable that the magnet 10 is moved automatically from the secure position 12 to the release position 13 and manually from the release position 13 to the secure position 12. The magnet 10 can be preloaded in the direction of the release position 13 by the preloading assembly 26. For example, it is possible that the locking assembly 24 first locks the magnet 10 in the secure position 12 and is unlocked. The magnet 10 is released and moved automatically from the secure position 12 to the release position 13. The drive unit 9 can now be disposed in the drive receptacle 20, for instance.

Alternatively, it is likewise possible that the magnet 10 is moved automatically from the secure position 12 to the release position 13 and from the release position 13 to the secure position. The automatic movement can be performed, for instance, by an electric motor and/or by the preloading assembly 26. For instance, the motor can be activated when the drive unit 9 is disposed in the drive receptacle 20 or is removed from the drive receptacle 20 and/or by a control command of a user. It is conceivable that the motor moves the magnet 10 from the secure position 12 to the release position 13, and the preloading assembly 26 moves the magnet 10 from the release position 13 to the secure position 12. Alternatively, it is likewise possible that the motor moves the magnet 10 from the release position 13 to the secure position 12, and the preloading assembly 26 moves the magnet 10 from the secure position 12 to the release position 13.

It can be provided that the magnet 10 is moved manually from the secure position 12 to the release position 13 when the drive unit 9 is received by the drive receptacle 20 of the portable container mounting 1, in particular is pushed or retracted into the drive receptacle 20, and that the magnet 10 is moved automatically from the release position 13 to the secure position 12 when the drive unit 9 is removed, in particular is extracted or pushed out, from the drive receptacle 20.

It can be provided that the magnet 10 is moved manually from the secure position 12 to the release position 13 once the drive unit 9 is received by the drive receptacle 20, and/or that the magnet 10 is moved from the release position 13 to the secure position 12 before the drive unit 9 is removed from the drive receptacle 20. The drive unit 9 can be received by the drive receptacle 20 in that the drive unit 9 and/or the portable container mounting 1 is moved in a sliding and/or pulling manner. The drive unit 9 can be removed from the drive receptacle 20 in that the drive unit 9 and/or the portable container mounting 1 is moved in a sliding and/or pulling manner.

1. A portable container mounting for use in a bioprocess for holding and transporting a single-use container which is filled with a fluid and has received a magnetically driven mixing element for mixing the fluid, wherein the portable container mounting has a container receptacle for receiving the single-use container having a drive location on which the mixing element is able to be disposed in the single-use container received in the container receptacle in such a manner that the mixing element is able to be driven by a drive unit on the drive location,
wherein the portable container mounting has a transport safeguard for securing and releasing the mixing element, which is disposed on the drive location, in its position, and in that the transport safeguard has a magnet which is movable between a defined secure position in which the mixing element is positionally secured, and a defined release position, in which the mixing element is positionally released.

2. The portable container mounting as claimed in claim 1, wherein the transport safeguard has a magnet mounting for holding the magnet in the secure position and the release position and in that the magnet is movable rotationally and/or translationally, by way of the magnet mounting from the secure position to the release position and/or from the release position to the secure position.

3. The portable container mounting as claimed in claim 1, wherein the transport safeguard has an operating assembly for operating the transport safeguard, and in that the magnet is movable manually and/or automatically by way of the operating assembly from the secure position to the release position and/or from the release position to the secure position.

4. The portable container mounting as claimed in claim 1, wherein the magnet is configured as a permanent magnet, and/or in that the magnet has a magnet clearance which, in the secure position of the magnet, at least partially surrounds the mixing element in the single-use container received in the container receptacle.

5. The portable container mounting as claimed in claim 1, wherein the container receptacle is formed from one or a plurality of container walls and one container base, and in that the drive location is disposed on the container base.

6. The portable container mounting as claimed in claim 1, wherein the magnet is disposed in the secure position on the drive location.

7. The portable container mounting as claimed in claim 1, wherein the portable container mounting has a drive receptacle for receiving the drive unit, which is disposed below the container receptacle, which drive receptacle is delimited upwardly by the container base of the container receptacle, and/or wherein the portable container mounting has a transport assembly having a plurality of transport rollers for displacing the portable container mounting.

8. A system for use in a bioprocess, wherein the system has a portable container mounting, having a container receptacle and a single-use container which has received a magnetically driven mixing element for mixing a fluid, wherein the single-use container is received in the container receptacle, the container receptacle has a drive location, and the mixing element is disposed in the single-use container received in the container receptacle on the drive location of the container receptacle in such a manner that the mixing element is able to be driven by a drive unit, wherein the portable container mounting has a transport safeguard for securing and releasing the mixing element, which is disposed on the drive location, in its position, and in that the transport safeguard has a magnet which is movable between a defined secure position in which the mixing element is positionally secured, and a defined release position, in which the mixing element is positionally released.

9. The system as claimed in claim 8, wherein the single-use container is configured as a flexible single-use container, and/or in that the mixing element has an agitator.

10. The system as claimed in claim 9, where the system has a drive unit, wherein the drive unit is configured separately from the portable container mounting and is able to be disposed on the drive location when required.

11. A method for holding and transporting a single-use container while using a portable container mounting, wherein the single-use container is received in a container receptacle of the portable container mounting, and a magnetically driven mixing element for mixing a fluid, which is received in the single-use container, is disposed on a drive location of the portable container mounting in such a manner that the mixing element is able to be driven by a drive unit,
wherein the mixing element disposed on the drive location is secured in its position by a transport safeguard of the portable container mounting, which transport safeguard has a magnet, in that the magnet is moved, in which the mixing element is positionally released, to a defined secure position in which the mixing element is positionally secured.

12. The method as claimed in claim 11, wherein the mixing element disposed on the drive location is positionally released by the transport safeguard of the portable container mounting in that the magnet is moved to a defined release position.

13. The method as claimed in claim 12, wherein the magnet is moved in a pivoting manner and/or translationally between the secure position and the release position.

14. The method as claimed in claim 11, wherein the magnet is moved from the secure position to the release position when a drive unit is received by a drive receptacle of the portable container mounting, and/or
in that the magnet is moved from the release position to the secure position when the drive unit is removed from the drive receptacle, and/or
in that the magnet is moved from the secure position to the release position when a drive unit is received by the drive receptacle, and/or
in that the magnet is moved from the release position to the secure position before a drive unit is removed from the drive receptacle.

15. The portable container mounting as claimed in claim 2, wherein the magnet mounting has a magnet guide for the guided movement of the magnet between the secure position and the release position, and in that the magnet guide has a guide bearing and a motion element which is mounted so as to be movable in the guide bearing.

16. The portable container mounting as claimed in claim 15, wherein the magnet mounting has a guide fastening for rigidly fastening the magnet guide to the container receptacle.

17. The portable container mounting as claimed in claim 3, wherein the operating assembly has an operating element and/or in that the operating assembly has a locking assembly for locking the magnet in the secure position and/or the release position, and/or in that the operating assembly has a preloading assembly for preloading the magnet to the secure position or the release position.

18. The portable container mounting as claimed in claim 4, wherein the magnet clearance is configured as a positioning aid for the easier positioning of the magnet on the drive location and/or about the mixing element.

19. The portable container mounting as claimed in claim 18, wherein the magnet clearance is configured in such a manner that the magnet has a cross section which is horizontal in particular in the secure position and represents substantially a circle, a segment of a circle, a semi-circle, a horseshoe, an ellipse or a partial ellipse.

20. The portable container mounting as claimed in claim 5, wherein the drive location has a drive clearance which extends over the entire thickness of the container base and in which the mixing element is able to be disposed at least partially or the drive unit is able to be disposed at least partially.
